(19) United States
(12) Patent Application Publication
(10) Pub. No.: US 2026/0069719 A1
(43) Pub. Date: Mar. 12, 2026

(54) RETROGRADE CORONARY VENOUS OR SINUS ADMINISTRATION OF THERAPEUTICS

(71) Applicants: TEMPLE UNIVERSITY OF THE COMMONWEALTH SYSTEM OF HIGHER EDUCATION, Philadelphia, PA (US); RENOVACOR, INC., Greenwich, CT (US)

(72) Inventors: FELDMAN Arthur M., Wynnewood, PA (US); MYERS Valerie, Arlington, VA (US); BOLOGNA Marcia, Leominster, MA (US)

(73) Assignees: TEMPLE UNIVERSITY OF THE COMMONWEALTH SYSTEM OF HIGHER EDUCATION, Philadelphia, PA (US); RENOVACOR, INC., Greewich, CT (US)

(21) Appl. No.: 18/682,875

(22) PCT Filed: Aug. 12, 2022
(86) PCT No.: PCT/US2022/074944

§ 371 Date: Feb. 9, 2024

Related U.S. Application Data
(60) Provisional application No. 63/260,225, filed on Aug. 12, 2021.
(60) Provisional application No. 63/263,442, filed on Nov. 2, 2021.
(60) Provisional application No. 63/343,985, filed on May 19, 2022.

(51) Int. Cl. A61K 48/00; A61K 38/17; C12N 15/86

(52) U.S. Cl. CPC A61K 48/0075; A61K 38/1709; A61K 48/0058; C12N 15/86; C12N 2750/14143

(57) ABSTRACT

The invention provides methods of delivering a therapeutic to the heart. In one embodiment, a method includes administering to a subject the therapeutic via retrograde coronary venous or sinus delivery thereby delivering the therapeutic to the heart.

### INTRODUCTION

Recent studies have shown that functional mutations in cardiac genes can result in the development of a dilated cardiomyopathy (DCM). While gene therapy has successfully ameliorated disease in non-cardiac familial conditions, attempts to correct gene expression in DCM have not been successful-raising the question of whether the heart can be successfully transduced using a viral vector.

### SUMMARY

Mutations in the BCL2-Associated Athanogene 3 (BAG3) gene have been associated with development of familial DCM, and patients may be able to be treated by cardiac transduction of wild type (WT) BAG3. In order to assess the efficacy of different routes of delivery to heart an antegrade delivery and retrograde delivery were studied in a large animal model.

Using a recombinant adeno-associated virus (rAAV) vector and selective catheterization of the left coronary artery for antegrade and distal to the great cardiac vein for retrograde delivery of a recombinant adeno-associated virus, serotype 2/9 (AAV2 ITR, AAV9 capsid; rAAV2/9)-BAG3 gene to Yucatan minipigs. Subsequent vector gene transduction was measured in various regions of the heart after administration of the rAAV vector.

In accordance with the invention, there are provided methods of delivering a therapeutic to the heart. In one embodiment, a method includes administering a therapeutic to a subject via retrograde coronary venous or sinus delivery thereby delivering the therapeutic to the heart.

In another embodiment, a method includes administering a therapeutic to a subject via retrograde coronary venous or sinus delivery, without occluding the left main coronary artery or without occluding antegrade flow, thereby delivering the therapeutic to the heart.

In another embodiment, a method includes administering a therapeutic to a subject via retrograde coronary venous or sinus delivery, with occluding the left main coronary artery or with occluding antegrade flow, thereby delivering the therapeutic to the heart.

In certain aspects, the therapeutic is delivered to the heart by a catheter positioned proximal to the coronary sinus and distal to the great cardiac vein.

In certain aspects, the therapeutic is delivered to the heart by a catheter positioned in or occluding the coronary sinus and distal to the origin of the great cardiac vein or the great cardiac vein.

In certain aspects, the protein comprises BCL2-Associated Athanogene 3 (BAG3).

In certain aspects, the nucleic acid comprises an expression vector.

In certain aspects, the expression vector comprises a viral vector, eukaryotic or yeast vector.

In certain aspects, the viral vector comprises an adeno-associated virus (AAV) vector, adenovirus vector, lentiviral vector or retroviral vector.

In certain aspects, the AAV vector comprises a capsid or inverted terminal repeat from any one of the following AAV serotypes: AAV1, AAV2, AAV3, AAV4, AAV5, AAV6, AAV7, AAV8, AAV9, AAV10, AAV11 or AAV12.

In certain aspects, the expression vector comprises a promoter functional in heart.

In certain aspects, the promoter is cardiac specific.

In certain aspects, the expression vector comprises a promoter functional in heart and a BAG3 polynucleotide or cDNA sequence.

In certain aspects, the subject is human.

In certain aspects, the subject or human is suffering from heart failure.

In certain aspects, the subject or human is suffering from heart failure with reduced ejection fraction.

In certain aspects, the subject or human is suffering from heart failure with preserved ejection fraction.

In certain aspects, the subject or human is suffering from familial dilated cardiomyopathy.

In certain aspects, the subject or human is suffering from non-familial dilated cardiomyopathy.

In certain aspects, the subject or human is suffering from ischemic heart disease or cardiomyopathy.

In certain aspects, the subject or human is suffering from nonischemic heart disease or cardiomyopathy.

In certain aspects, the subject or human is at risk of ischemia/reperfusion injury.

In certain aspects, the subject or human is scheduled for or is a candidate for a vascular interventional or medical procedure that could be to ischemia/reperfusion injury.

In certain aspects, the vascular interventional or medical procedure comprises a procedure using a catheter, a stent, angioplasty, bypass surgery or coronary artery bypass graft.

In certain aspects, the subject or human is scheduled for or is a candidate for peripheral vascular disease surgery.

In certain aspects, the subject or human has a mutation in their endogenous BAG3 polynucleotide or polypeptide.

In certain aspects, the subject or human has reduced expression or activity of endogenous BAG3 polynucleotide or polypeptide.

In certain aspects, the therapeutic is administered to the subject or human via the great cardiac vein.

In certain aspects, the therapeutic is administered via a catheter positioned proximal but distal to the great cardiac vein.

In certain aspects, the therapeutic is delivered into the great cardiac vein, left azygous or any veins that feed off the coronary sinus.

In certain aspects, the therapeutic is administered via infusion for a time up to about 20 minutes.

In certain aspects, the therapeutic is administered at a rate of about 1 ml per minute to 5 ml per minute.

In certain aspects, the therapeutic is administered at a rate of about 1 ml per minute.

In certain aspects, the therapeutic is administered in a volume from about 10 ml to about 100 mL.

In certain aspects, the therapeutic is administered in a volume from about 20 ml to about 75 mL.

In certain aspects, the therapeutic is administered in a volume from about 30 ml to about 60 mL.

In certain aspects, the therapeutic is administered in a volume from about 40 ml to about 50 mL.

In certain aspects, the therapeutic is delivered to and/or expressed in one or more of anterior left ventricle, anterior lateral left ventricle, inferior left ventricle, inferior lateral ventricle, septum or right ventricle.

In certain aspects, the therapeutic is expressed throughout the heart.

In certain aspects, the therapeutic reduces one or more symptoms of a cardiac disease.

In certain aspects, the therapeutic improves cardiac function or cardiac contractility.

In certain aspects, the therapeutic increases left ventricle ejection fraction.

In certain aspects, a therapeutic, such as a viral vector is administered or used.

In certain aspects, a viral vector is administered or used at a dose from about 1×10¹¹ vg/kg to about 1.0×10¹⁴ vg/kg.

In certain aspects, a viral vector is administered at a dose from about 1.0×10¹² vg/kg to about 0.5×10¹⁴ vg/kg.

In certain aspects, a viral vector is administered at a dose from about 3.0×10¹² vg/kg to about 1.0×10¹³ vg/kg.

In certain aspects, a viral vector is administered at a dose from about 3.0×10¹² vg/kg to about 9.0×10¹² vg/kg.

In certain aspects, a viral vector is administered at a dose from about 3.0×10¹² vg/kg to about 8.0×10¹² vg/kg.

In certain aspects, a viral vector is administered at a dose from about 3.0×10¹² vg/kg to about 5.0×10¹² vg/kg.

### DESCRIPTION OF THE DRAWINGS

FIG. 1A shows coronary retrograde administration.

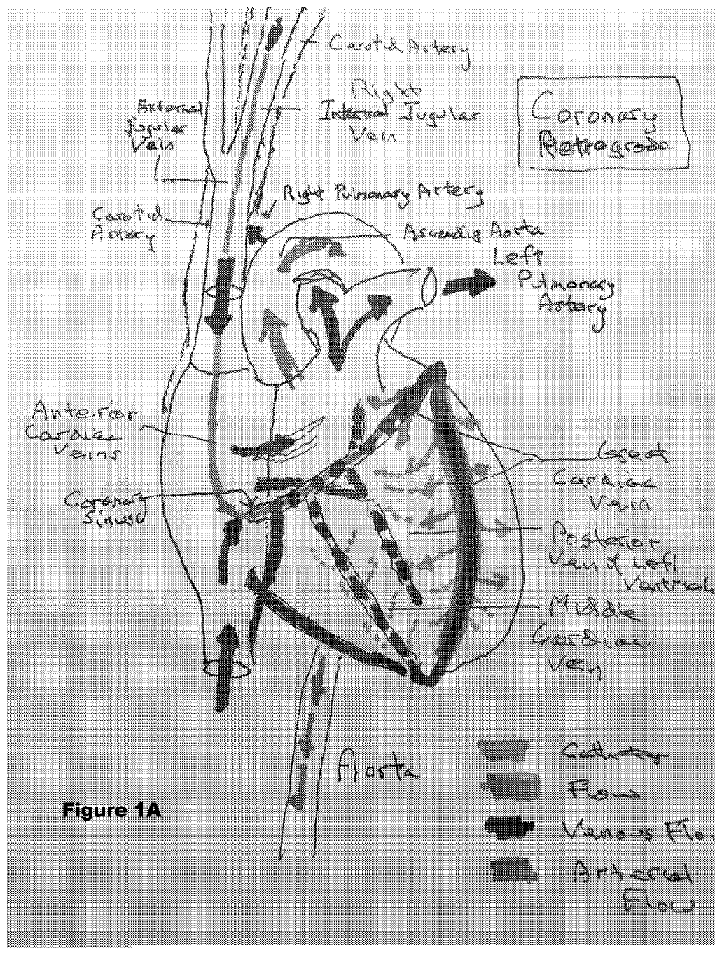

FIG. 1B shows coronary antegrade administration.

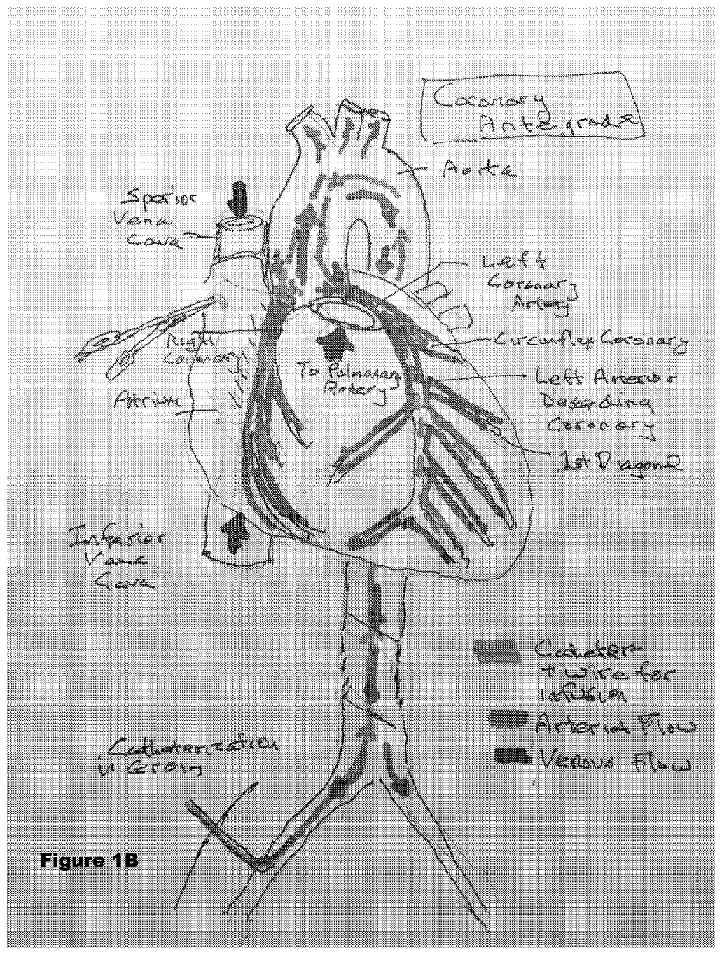

FIG. 1C shows retrograde coronary sinus administration.

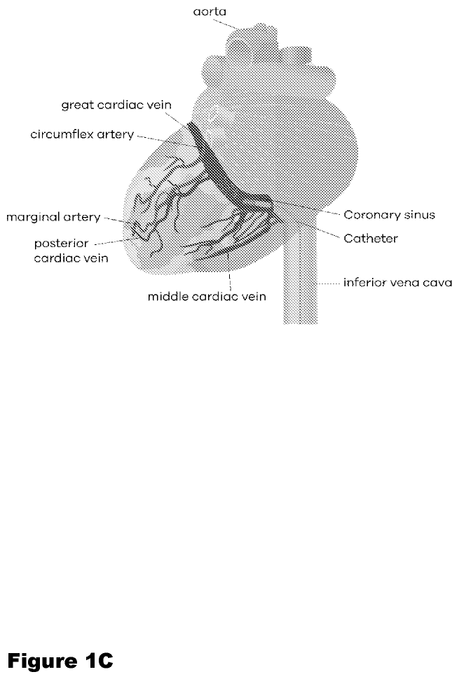

FIG. 2 shows Western blot for GFP (27 kDa) in select samples from an initial study comparing retrograde and antegrade dosing in a large animal model. GFP is expressed in LV after retrograde dosing. In contrast, no expression of GFP was detected in LV after antegrade dosing.

FIG. 3 shows ≥1 vector genome (vg) copy numbers per cardiomyocyte in most regions. Broad expression throughout heart sections with retrograde coronary sinus infusion (RCSI). Vehicle (Not visible on graph), 5e13vg (low dose), 1e14vg (medium dose), 2.5e14vg (high dose).

FIG. 4 illustrates distribution of vector genomes (vg) across the myocardium in the coronary sinus of the pig after retrograde infusion of AAV9-BAG3, after applying the exclusion criteria: any result more than 3 standard deviations from the mean for each individual animal was excluded. Applying this criteria resulted in the exclusion of 3 values, each animal had 1 tissue sections assessed per heart, there were 8 animals in the study so 3/144 samples met the exclusion criteria. Mean SEM vg per myocyte. Measurements from tissues obtained from 5 left ventricular regions. Anterior (AN), anterior lateral (AL), inferior lateral (IL), inferior (IN), septum(S) and right ventricle (RV).

FIG. 5 illustrates nomenclature used for labeling heart rings and regions. Samples are either snap frozen or formalin fixed.

FIG. 6 shows heart biodistribution of BAG3 administered via retrograde coronary sinus infusion (RCSI). BAG3 is broadly distributed and there was dose dependent, strong and diffuse BAG3 transduction. Mean VCNs of ˜1, 3, and 6 (low-, mid-, and high-dose cohorts respectively; low=1e14vg, mid=2.5e14 and high=5e14vg per animal).

FIG. 7 shows broad BAG3 distribution in heart. One-way ANOVA Post-hoc: Tukey's test *=p≤0.05, **=p≤0.01, ***=p≤0.001, ****=p≤0.0001.

FIG. 8A shows dose dependent transduction of BAG3 into the heart. One-way ANOVA Post-hoc: Tukey's test *=p≤0.05, **=p≤0.01, ***=p≤0.001, ****=p≤0.0001

FIG. 8B shows dose dependent transduction of BAG3 into the heart. One-way ANOVA Post-hoc: Tukey's test *=p≤0.05, **=p≤0.01, ***=p≤0.001, ****=p≤0.0001

FIG. 8C shows dose dependent transduction of BAG3 into the heart. One-way ANOVA Post-hoc: Tukey's test *=p≤0.05, **=p≤0.01, ***=p≤0.001, ****=p≤0.0001

### DETAILED DESCRIPTION

The invention provides methods of delivering a therapeutic to the heart. In one embodiment, a method includes administering to a subject the therapeutic via retrograde coronary venous or sinus delivery thereby delivering the therapeutic to the heart.

The invention also provides methods of delivering a nucleic acid or protein (or both) to the heart. In one embodiment, a method includes administering to a subject the nucleic acid or protein via retrograde coronary venous or sinus delivery thereby delivering the nucleic acid or protein to the heart.

The invention also provides methods of delivering an expression vector to the heart. In one embodiment, a method includes administering to a subject the expression vector via retrograde coronary venous or sinus delivery thereby delivering the expression vector to the heart.

The invention also provides methods of delivering a viral vector to the heart. In one embodiment, a method includes administering to a subject the viral vector via retrograde coronary venous delivery thereby delivering the viral vector to the heart.

Viral vectors that may be used in the invention methods and uses. In certain embodiments, viral vectors that may be used in the invention include, for example and without limitation, retroviral, adeno associated virus (AAV),

Bcl-2 associated anthanogene-3 (BAG3), also known as BCL2-Associated Athanogene 3; MFM6; Bcl-2-Binding Protein Bis; CAIR-1; Docking Protein CAIR-1; BAG Family Molecular Chaperone Regulator 3; BAG-3; BCL2-Binding Athanogene 3; or BIS, is a cytoprotective polypeptide that competes with Hip-1 for binding to HSP 70. The NCBI reference amino acid sequence for BAG3 can be found at Genbank under accession number NP_004272.2; Public GI:14043024. The amino acid sequence of Genbank accession number NP_004272.2; Public GI:14043024 is referred to herein as SEQ ID NO: 1. The NCBI reference nucleic acid sequence for BAG3 can be found at Genbank under accession number NM_004281.3 GI:62530382. The nucleic acid sequence of Genbank accession number NM_004281.3 GI:62530382 is referred as SEQ ID NO: 2. Other BAG3 amino acid sequences include, for example, without limitation, 095817.3 GI:12643665 (SEQ ID NO: 3); EAW49383.1 GI:119569768 (SEQ ID NO: 4); EAW49382.1 GI:119569767 (SEQ ID NO: 5); and CAE55998.1 GI:38502170 (SEQ ID NO: 6). The BAG3 polypeptide of the invention can be a can be a variant of a polypeptide described herein, provided it retains functionality.

As used herein, an “agent” is meant to encompass any molecule, chemical entity, composition, drug, therapeutic agent, or biological agent or entity capable of preventing, ameliorating, or treating a disease, disorder or other medical condition. The term includes small molecule compounds, antisense reagents, siRNA, reagents, antibodies, enzymes, peptides organic or inorganic molecules, natural or synthetic compounds, cells and organelles, such as mitochondria. An agent can be assayed in accordance with the methods and uses of the invention at any stage during clinical trials, during pre-trial testing, or following FDA-approval.

The terms “polypeptides,” “proteins” and “peptides” are used interchangeably herein. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. Such polypeptides, proteins and peptides encoded by the polynucleotide sequences can be but are not required to be identical to an endogenous protein in the treated patient.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA).

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” polynucleotide or nucleic acid sequence refers to a polynucleotide inserted into a plasmid or vector for purposes of vector mediated transfer/delivery of the polynucleotide into a cell. Heterologous nucleic acid sequences are distinct from viral nucleic acid, i.e., are non-native with respect to viral nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

The term “expression vector” as used herein refers to a vector containing a nucleic acid sequence (e.g., BAG3) coding for all or at least part of a gene capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

An expression control or regulatory element confers or enhances transcription of a polynucleotide or nucleic acid sequence in an expression vector. Expression control elements include but are not limited to, for example, promoters and enhancers.

A “promoter” as used herein can refer to a DNA sequence that is typically located adjacent to a nucleic acid sequence (e.g., BAG3). A promoter typically increases an amount of nucleic acid sequence (e.g., BAG3) expressed compared to an amount expressed when no promoter exists.

An “enhancer” as used herein can refer to a sequence that is located adjacent to the nucleic acid sequence (e.g., BAG3). Enhancer elements are typically located upstream of a promoter element but also function and can be located downstream of or within a nucleic acid sequence (e.g., BAG3). Hence, an enhancer element can be located 100 base pairs, 200 base pairs, or 300 or more base pairs upstream or downstream of a nucleic acid sequence (e.g., BAG3). Enhancer elements typically increase expression of a nucleic acid sequence (e.g., BAG3) above increased expression afforded by a promoter element.

Examples of expression control elements or expression regulatory elements that can be used in methods and uses according to the invention, include, for example and without limitation, cytomegalovirus (CMV) immediate early promoter/enhancer, Rous sarcoma virus (RSV) promoter/enhancer, SV40 promoter, dihydrofolate reductase (DHFR) promoter, chicken β-actin (CBA) promoter, phosphoglycerol kinase (PGK) promoter, and elongation factor-1 alpha (EF1-alpha) promoter.

In certain embodiments, viral vectors that may be used in the invention methods and uses. In certain embodiments, viral vectors that may be used in the invention include, for example and without limitation, retroviral, adeno associated virus (AAV), adenoviral, helper-dependent adenoviral, hybrid adenoviral, herpes simplex virus, lentiviral, poxvirus, Epstein-Barr virus, vaccinia virus, and human cytomegalovirus vectors, including recombinant versions thereof.

The term “recombinant,” as a modifier of a viral vector, such as a recombinant AAV (rAAV) vector, as well as a modifier of sequences such as recombinant polynucleotides and polypeptides, means that compositions have been manipulated (i.e., engineered) in a fashion that generally does not occur in nature. A “recombinant viral vector” therefore refers to a viral vector comprising one or more heterologous gene products or sequences.

Since many viral vectors exhibit size-constraints associated with packaging, the heterologous gene products or sequences are typically introduced by replacing one or more portions of the viral genome. Such viruses may become replication-defective, requiring the deleted function(s) to be provided in trans (i.e., “helper” function) during viral replication and encapsidation (by using, e.g., a helper virus or a packaging cell line carrying gene products necessary for replication and/or encapsidation, such as AAV rep, AAV cap, human adenoviral E4 and adenoviral VA RNA). Modified viral vectors in which a polynucleotide to be delivered is carried on the outside of the viral particle have also been described (see, e.g., Curiel, D T, et al., PNAS 88:8850-8854, 1991).

A particular example of a recombinant adeno associated virus (rAAV) vector would be where a nucleic acid that is not normally present in a wild-type AAV genome (heterologous polynucleotide) is inserted within the AAV genome. An example of which would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to an AAV vector, as well as sequences such as polynucleotides, recombinant forms including AAV vectors, polynucleotides, etc., are expressly included in spite of any such omission.

A “rAAV vector,” for example, is derived from a wild-type genome of AAV by using molecular methods to remove all or a part of a wild-type AAV genome, and replacing with a non-native (heterologous) nucleic acid, such as a nucleic acid encoding a therapeutic protein or polynucleotide sequence. Typically, for a rAAV vector one or both inverted terminal repeat (ITR) sequences of AAV genome are retained. A rAAV is distinguished from an AAV genome since all or a part of an AAV genome has been replaced with a non-native sequence with respect to the AAV genomic nucleic acid, such as with a heterologous nucleic acid encoding a therapeutic protein or polynucleotide sequence. Incorporation of a non-native (heterologous) sequence therefore defines an AAV as a “recombinant” AAV vector, which can be referred to as a “rAAV vector.”

A recombinant AAV vector sequence (or genome) can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion.” Such rAAV, rAAV particles and rAAV virions include proteins that encapsidate or package a vector genome. Particular examples include in the case of AAV, capsid proteins.

A “vector genome,” which may be abbreviated as “vg,” refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV may cross-react with one or more other AAV serotypes due to homology of capsid protein sequence.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors include any viral strain or serotype. For example and without limitation, a rAAV vector genome or particle (capsid, such as VP1, VP2 and/or VP3) can be based upon any AAV serotype, such as AAV-1, -2, -3, -4, -5, -6, -7, -8, -9, -10, -11, -12, AAV3B or AAV-2i8, for example. Such vectors can be based on the same strain or serotype (or subgroup or variant), or be different from each other. For example and without limitation, a rAAV plasmid or vector genome or particle (capsid) based upon one serotype genome can be identical to one or more of the capsid proteins that package the vector. In addition, a rAAV plasmid or vector genome can be based upon an AAV serotype genome distinct from one or more of the capsid proteins that package the vector genome, in which case at least one of the three capsid proteins could be a different AAV serotype, e.g., AAV1, AAV2, AAV3, AAV3B, AAV-2i8 (AAV2/AAV8 chimera), AAV4, AAV5, AAV6, AAV7, AAV8, AAV9, AAV10, AAV11, AAV12, or variant thereof, for example. More specifically, a rAAV2 vector genome can comprise AAV2 ITRs but capsids from a different serotype, such as AAV1, AAV3, AAV3B, AAV4, AAV5, AAV6, AAV7, AAV8, AAV9, AAV10, AAV11, AAV12, AAV-2i8, or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV3B, AAV-2i8, AAV4, AAV5, AAV6, AAV7, AAV8, AAV9, AAV10, AAV11, or AAV12 capsid proteins (VP1, VP2, and/or VP3 sequences). In certain embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV3B, AAV4, AAV5, AAV6, AAV7, AAV8, AAV9, AAV10, AAV11, or AAV12 ITR(s).

In certain embodiments, rAAV vectors include AAV1, AAV2, AAV3, AAV3B, AAV-2i8, AAV4, AAV5, AAV6, AAV7, AAV8, AAV9, AAV10, AAV11, or AAV12 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV, such as AAV1, AAV2, AAV3, AAV3B, AAV-2i8, AAV4, AAV5, AAV6, AAV7, AAV8, AAV9, AAV10, AAV11, AAV12 and variants, hybrids and chimeric sequences, can be constructed using recombinant techniques that are known to a skilled artisan, to include one or more heterologous polynucleotide sequences (transgenes) flanked with one or more functional AAV ITR sequences. Such AAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

In certain embodiments, a lentivirus used in the invention may be a human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), or caprine arthritis encephalitis virus (CAEV). Lentiviral vectors are capable of providing efficient delivery, integration and long-term expression of heterologous polynucleotide sequences into non-dividing cells both in vitro and in vivo. A variety of lentiviral vectors are known in the art, see Naldini et al. (Proc. Natl. Acad. Sci. USA, 93:11382-11388 (1996); Science, 272:263-267 (1996)), Zufferey et al., (Nat. Biotechnol., 15:871-875, 1997), Dull et al., (J Virol. 1998 November; 72 (11): 8463-71, 1998), U.S. Pat. Nos. 6,013,516 and 5,994,136, any of which may be a suitable viral vector for use in the invention.

Recombinant viral vector doses can be formulated, administered or delivered at any appropriate dose. Generally, doses will range from at least 1×10⁸ vector genomes per kilogram (vg/kg), or more, for example, 1×10⁹, 1×10¹⁰, 1×10¹¹, 1×10¹², 1×10¹³ or 1×10¹⁴, or more, vector genomes per kilogram (vg/kg) of the weight of the patient, to achieve an effect. rAAV doses in the range of 1×10¹⁰-1×10¹¹ vg/kg in mice, and 1×10¹²-1×10¹³ vg/kg in dogs have been effective. More particularly, a dose from about 1×10¹¹ vg/kg to about 5×10¹⁴ vg/kg inclusive, or from about 5×10¹¹ vg/kg to about 1×10¹⁴ vg/kg inclusive, or from about 5×10¹¹ vg/kg to about 5×10¹³ vg/kg inclusive, or from about 5×10¹¹ vg/kg to about 1×10¹³ vg/kg inclusive, or from about 5×10¹¹ vg/kg or about 5×10¹² vg/kg inclusive, or from about 5×10¹¹ vg/kg to about 1×10¹² vg/kg inclusive. Doses can be, for example, about 5×10¹⁴ vg/kg, or less than about 5×10¹⁴ vg/kg, such as a dose from about 2×10¹¹ to about 2×10¹⁴ vg/kg inclusive, in particular, for example, about 2×10¹² vg/kg, about 6×10¹² vg/kg, or about 2×10¹³ vg/kg.

An “effective amount,” “sufficient amount” or “therapeutically effective amount” refers to an amount that provides, in single or multiple doses, alone or in combination, with one or more other compositions, treatments, protocols, or therapeutic regimens agents, a detectable response of any duration of time (long or short term), an expected or desired outcome in or a benefit to a patient of any measurable or detectable degree or for any duration of time (e.g., for minutes, hours, days, months, years, or cured). The doses of an “effective amount” or “sufficient amount” for treatment of the condition, disorder or disease (e.g., to ameliorate or to provide a therapeutic benefit or improvement of the condition, disorder or disease) typically are effective to provide a response to one, multiple or all adverse symptoms, consequences or complications of the disease, one or more adverse symptoms, disorders, illnesses, pathologies, or complications of the condition, disorder or disease, for example, caused by or associated with the condition, disorder or disease, to a measurable extent, although decreasing, reducing, inhibiting, suppressing, limiting or controlling progression or worsening of the condition, disorder or disease is a satisfactory outcome.

An effective amount or a sufficient amount can but need not be provided in a single formulation or administration, may require multiple administrations, and can but need not be, administered alone or in combination with another composition (e.g., agent), treatment, protocol or therapeutic regimen. For example, the amount may be proportionally increased as indicated by the need of the patient, type, status and severity of the condition, disorder or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given patient. Amounts considered effective also include amounts that result in a reduction of the use of another treatment, therapeutic regimen or protocol.

An effective amount or a sufficient amount need not be effective in each and every patient treated, nor a majority of treated patients in a given group or population. An effective amount or a sufficient amount means effectiveness or sufficiency in a particular patient, not a group or the general population. As is typical for such methods, some patients will exhibit a greater response, or less or no response to a given treatment method or use.

Methods, uses and formulations of the invention therefore include providing a detectable or measurable beneficial effect to a patient, or any objective or subjective transient or temporary, or longer-term improvement (e.g., cure) in the condition, disorder or disease. Thus, a satisfactory clinical endpoint is achieved when there is an incremental improvement in the patient's condition, disorder or disease or a partial reduction in severity, frequency, duration or progression of one or more associated adverse symptoms or complications of the condition, disorder or disease, or inhibition, reduction, elimination, prevention or reversal of one or more of the physiological, biochemical or cellular manifestations or characteristics of the condition, disorder or disease. A therapeutic benefit or improvement (“ameliorate” is used synonymously) therefore need not be complete ablation of any or all adverse symptoms or complications associated with the condition, disorder or disease but is any measurable or detectable, objectively or subjectively, meaningful improvement in the condition, disorder or disease. For example, inhibiting a worsening or progression of the condition, disorder or disease, or an associated symptom (e.g., slowing progression or stabilizing one or more symptoms, complications or physiological or psychological effects or responses), even if only for a few days, weeks or months, even if complete ablation of the condition, disorder or disease, or an associated adverse symptom is not achieved is considered to be a beneficial effect.

“Treatment” is an intervention performed with the intention of preventing the development, altering the pathology or one or more symptoms of a condition, disorder or disease or delaying progression or worsening of a condition, disorder or disease. Accordingly, “treatment” refers to both therapeutic treatment and prophylactic or preventative measures. “Treatment” may also be specified as palliative care.

“Prophylaxis” and grammatical variations thereof mean a method in accordance with the invention in which contact, administration or in vivo delivery to a subject is prior to manifestation or onset of a condition, disorder or disease (or an associated symptom or physiological or psychological response), such that it can eliminate, prevent, inhibit, decrease or reduce the probability, susceptibility, onset or frequency of having a condition, disorder or disease, or an associated symptom. Target patients for prophylaxis can be one of increased risk (probability or susceptibility) of contracting a condition, disorder or disease, such as heart failure, heart failure with reduced ejection fraction, heart failure with preserved ejection fraction, or an associated symptom, or recurrence of a previously diagnosed condition, disorder or disease, or an associated symptom, as set forth herein.

Those in need of treatment include those already with the condition, disorder or disease as well as those in which the condition, disorder or disease is to be prevented. Accordingly, “treating” or “treatment” of condition, disorder or disease includes; (1) preventing or delaying the appearance of clinical symptoms of the condition, disorder or disease developing in a human or other mammal that may be afflicted with or predisposed to the condition, disorder or disease but does not yet experience or display clinical or subclinical symptoms of the condition, disorder or disease; (2) inhibiting the condition, disorder or disease, i.e., arresting, reducing or delaying the development of the condition, disorder or disease or a relapse thereof (in case of maintenance treatment) or at least one clinical or subclinical symptom thereof; or (3) relieving the disease, i.e., causing regression of the condition, disorder or disease or at least one of its clinical or subclinical symptoms. The benefit to a patient to be treated is either statistically significant or at least perceptible to the patient or to the physician.

The term “ameliorate” means a detectable or measurable improvement in a patient's condition, disorder or disease or symptom thereof, or an underlying cellular response. A detectable or measurable improvement includes a subjective or objective decrease, reduction, inhibition, suppression, limit or control in the occurrence, frequency, severity, progression, or duration of the condition, disorder or disease, or complication caused by or associated with the condition, disorder or disease, or an improvement in a symptom or an underlying cause or a consequence of the condition, disorder or disease, or a reversal of the condition, disorder or disease.

Formulations can be administered one from one or more times per day; once every other day; one or more times per week; one or more times per month; one or more times per year; or 1-2 times over the patient's lifetime. The skilled artisan will appreciate that certain factors can influence the dosage and timing required to treat a patient, including but not limited to the severity of the condition, disorder or disease, desired outcome, previous treatments, the general health and/or age of the patient, and other diseases present. Moreover, treatment of a patient with a therapeutically effective amount in accordance with the invention can include a single treatment or multiple treatments, such as a series of treatments.

Formulations, compositions and pharmaceutical compositions of the invention include compositions wherein the active agent is contained in an effective amount to achieve the intended therapeutic purpose. Determining an effective dose is well within the capability of a skilled medical practitioner using techniques and guidance known in the art and using the teachings provided herein.

Formulations, such as pharmaceutical compositions, may be delivered to a patient, so as to allow nucleic acid transcription and translation of encoded protein. In certain embodiments, formulations, such as pharmaceutical compositions, comprise sufficient genetic material to enable production of a therapeutically effective amount of BAG3 in the patient to treat a condition, disorder or disease.

By the term “modulate,” it is meant that any of the mentioned activities of the compounds embodied herein, are, e.g., increased, enhanced, increased, promoted, agonized (acts as an agonist), decreased, reduced, inhibited, suppressed, blocked or antagonized (acts as an antagonist). Modulate can reduce or decrease its activity below baseline values, e.g., a reduction or decrease of 1 to 5 fold, 1 to 10 fold, 5 to 10 fold, 10 to 20 fold, 20 to 30 fold, 40 to 50 fold, etc., or at least 1-fold, 2-fold, 3-fold, 5-fold, 10-fold, 20 fold, 50 fold 100-fold, etc. Modulate also can increase or enhance activity over baseline values, e.g., an increase or enhancement of 1 to 5 fold, 1 to 10 fold, 5 to 10 fold, 10 to 20 fold, 20 to 30 fold, 40 to 50 fold, etc., or at least 1-fold, 2-fold, 3-fold, 5-fold, 10-fold, 20 fold, 50 fold 100-fold, etc.

Invention methods and uses can be used in primate (e.g., human) and veterinary medical applications. Suitable patients therefore include mammals, such as humans, as well as non-human mammals. Suitable patients include mammals, such as humans, in need of or that would benefit from BAG3 expression or increasing BAG3 activity.

The terms “patient” and “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human patients include fetal, neonatal, infant, juvenile and adult subjects. Patients also include animal disease models, for example, mouse and other animal models of BAG3 insufficiency.

Compositions and formulations may be sterile and methods and uses may be practiced using sterile technique, and optionally with sterile compositions and formulations. Compositions may be formulated with or be administered in any biocompatible pharmaceutical carrier, including, but not limited to, saline, buffered saline, dextrose, and water. The compositions may be formulated, administered or delivered to a patient alone, or in combination with other agents, which influence dosage amount, administration frequency and/or therapeutic efficacy.

Formulations, methods and uses of the invention include delivery and administration systemically, regionally or locally (e.g., to a particular region, tissue, organ or cell), or by any route, for example, by injection or infusion. Administration or delivery of the compositions, formulations and pharmaceutical compositions in vivo may generally be accomplished via injection using a conventional syringe or catheter, although other delivery methods are envisioned. For example, formulations and compositions may be administered to a patient via retrograde coronary venous or sinus delivery.

Also in accordance with the invention, nucleic acids, expression vectors including viral vectors and viral particles may be encapsulated or complexed with liposomes, nanoparticles, lipid nanoparticles, polymers, microparticles, microcapsules, micelles, or extracellular vesicles.

A “lipid nanoparticle” or “LNP” refers to a lipid-based vesicle useful for administration or delivery of nucleic acids, expression vectors including viral vectors having dimensions on the nanoscale, i.e., from about 10 nm to about 1000 nm, or from about 50 to about 500 nm, or from about 75 to about 127 nm. Without being bound by theory, LNP is believed to provide nucleic acid, expression vector or recombinant viral vector with partial or complete shielding from the immune system. Shielding allows delivery of the nucleic acid, expression vector or viral vector to a tissue or cell while avoiding inducing a substantial immune response against the nucleic acid, expression vector or viral vector in vivo. Shielding may also allow repeated administration without inducing a substantial immune response. Shielding may also improve or increase delivery efficiency, duration of therapeutic effect and/or therapeutic efficacy in vivo.

The AAV surface carries a slight negative charge. As such it may be beneficial for the LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018, 187, 8,999,351, 8,722,082, 8,642,076, 8,569,256, 8,466, 122, and 7,745,651 and U.S. Patent Publication Nos. 2016/0213785, 2016/0199485, 2015/0265708, 2014/0288146, 2013/0123338, 2013/0116307, 2013/0064894, 2012/0172411 and 2010/0117125.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids may also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

In certain embodiments, cationic lipid may be present in an amount from about 10% by weight of the LNP to about 85% by weight of the lipid nanoparticle, or from about 50% by weight of the LNP to about 75% by weight of the LNP.

LNP can comprise a neutral lipid. Neutral lipids may comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component may be a lipid having two acyl groups (e.g., diacylphosphatidylcholine and diacylphosphatidylethanolamine).

In certain embodiments, the neutral lipid may be present in an amount from about 0.1% by weight of the lipid nanoparticle to about 75% by weight of the LNP, or from about 5% by weight of the LNP to about 15% by weight of the LNP.

A biological sample is typically obtained from or produced by a biological organism. Examples of biological samples from a patient that may be analyzed include, for example and without limitation, whole blood, serum, plasma, the like, and a combination thereof. Other biological samples from a patient include, for example and without limitation, cerebrospinal fluid or simply spinal fluid. A biological sample may be devoid of cells, or may include cells (e.g., red blood cells, platelets and/or lymphocytes).

The invention provides compositions, such as kits, that include packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, virus (e.g., AAV, lentivirus) vector, or virus particle.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a condition, disorder or disease for which a kit component may be used. Labels or inserts can include instructions for the clinician or patient for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the patient or clinician regarding situations where it would not be appropriate to use a particular composition. Adverse side effects or complications could also occur when the patient has, will be or is currently taking one or more other medications that may be incompatible with the composition, or the patient has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component.

Unless otherwise defined, all technical and scientific terms used herein have the same meaning as commonly understood by one of ordinary skill in the art to which the invention belongs. Although methods and materials similar or equivalent to those described herein can be used in the practice or testing of the invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses and particles.

As used herein, the terms “comprising,” “comprise” or “comprised,” and variations thereof, in reference to defined or described elements of an item, composition, formulation, method, process, system, etc. are meant to be inclusive or open ended, permitting additional elements, thereby indicating that the defined or described item, composition, formulation, method, process, system, etc. includes those specified elements—or, as appropriate, equivalents thereof—and that other elements can be included and still fall within the scope/definition of the defined item, composition, formulation, method, process, system, etc.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, or up to 10%, or up to 5% within a given value. Alternatively, particularly with respect to biological systems or processes, the term can mean within an order of magnitude, for example within 5-fold, 4-fold, 3-fold, 2-fold, or within 1-fold, of a given value. Where particular values are described in the application and claims, unless otherwise stated the term “about” meaning within an acceptable error range for the particular value should be understood.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “1×10¹¹ vg/kg to about 1.0×10¹⁴ vg/kg” body weight of a patient includes 1.1×10¹¹ vg/kg, 1.2×10¹¹ vg/kg, 1.3×10¹¹ vg/kg, 1.4×10¹¹ vg/kg, 1.5×10¹¹ vg/kg, etc., as well as 0.9×10¹⁴ vg/kg, 0.8×10¹⁴ vg/kg, 0.7×10¹⁴ vg/kg, 0.6×10¹⁴ vg/kg, 0.5×10¹⁴ vg/kg, etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a recombinant viral vector “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days 81 days, 82 days, 83 days, 84 days, 85 days, etc., and so forth.

“Optional” or “optionally” means that the subsequently described circumstance may or may not occur, such that the description includes instances where the circumstance occurs and instances where it does not.

The invention is generally disclosed herein using affirmative language to describe the numerous embodiments of the invention. The invention also specifically includes embodiments in which particular subject matter is excluded, in full or in part, such as compositions or formulations, uses, method steps and conditions, protocols, or procedures. For example, in certain embodiments of the invention, compositions and/or method steps are excluded. Thus, even though the invention is generally not expressed herein in terms of what the invention does not include, aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

An initial assessment of delivery method was performed using AAV9 with a reporter gene (GFP). This study was done using two AAV doses, 1e13vg and 5e13vg. The western blotting was performed by a CRO (Absorption Systems) on one sample from the left ventricle, one from the right ventricle, and one from the septum. On this blot, tissue from a mouse dosed retro orbitally with 1e13vg of AAV9-GFP was used as a control.

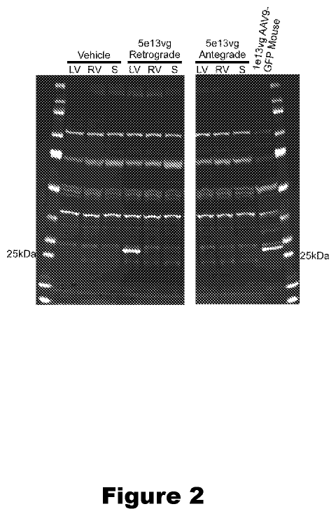

Results of this study (FIG. 2) showed clear superiority of the retrograde delivery method in animals dosed with 5e13vg.

### Example 2

rAAV2/9-BAG3 Experimental Design:

| | | Dose | Dose | Dose | Main Study | |
|---|---|---|---|---|---|---|
| Group | Test | Level | Volume | Concentration | No. of | Necropsy |
| No. | Material | (vg) | (mL) | (vg/mL) | Males | Interval |
| 1 | Vehicle - 80 mL | 0 | 80 | 0 | 1 | Week 8 |
| | at 5 mL/min | | | | | |
| 2 | Efficacious | 5e13 | 40 | 1.25e12 | 2 | |
| | Dose | | | | | |
| | AAV9BAG3 - | | | | | |
| | 40 ml at | | | | | |
| | 2 ml/min | | | | | |
| 3 | Efficacious | 5e13 | 80 | 6.25e11 | 2 | |
| | Dose | | | | | |
| | AAV9BAG3 | | | | | |
| | 80 ml at | | | | | |
| | 5 ml/min | | | | | |
| 4 | Mid-dose | 1e14 | 40 | 2.5e12 | 1 | |
| | AAV9BAG3 | | | | | |
| | 40 mL at | | | | | |
| | 2 mL/min | | | | | |
| 5 | Mid-dose | 1e14 | 80 | 1.25e12 | 1 | |
| | AAV9BAG3 | | | | | |
| | 80 mL at | | | | | |
| | 5 mL/min | | | | | |
| 6 | High Dose | 2.5e14 | 80 | 3.125e12 | 1 | |
| | AAV9BAG3 | | | | | |
| | 80 ml at | | | | | |
| | 5 ml/min | | | | | |

No. = Number;
vg = vector genomes

Route: Retrograde coronary sinus infusion (RCSI)
Frequency: Once on Day 1
Dose Level: 5e13vg (1,219 μl), 1e14vg (2,439 μl) and 2.5e14 vg (6,097 μl)

Methods: Healthy male Yucatan minipigs (weight: 30-40 kg) were sedated and randomized to receive one of 3 doses (5e13vg (A, n=4), 1e14vg (B, n=2), 2.5e14vg (C, n=1) of rAAV2/9-BAG3 (vg=rAAV vector genomes) through the tip of a catheter placed under fluoroscopy in the coronary sinus (CS). Occlusion of the CS was confirmed by venography prior to injection. AAV9-BAG3 vector was infused in 40-80 mL of vehicle over 20 minutes between 2 and 5 mL/min with simultaneous balloon occlusion at the ostium.

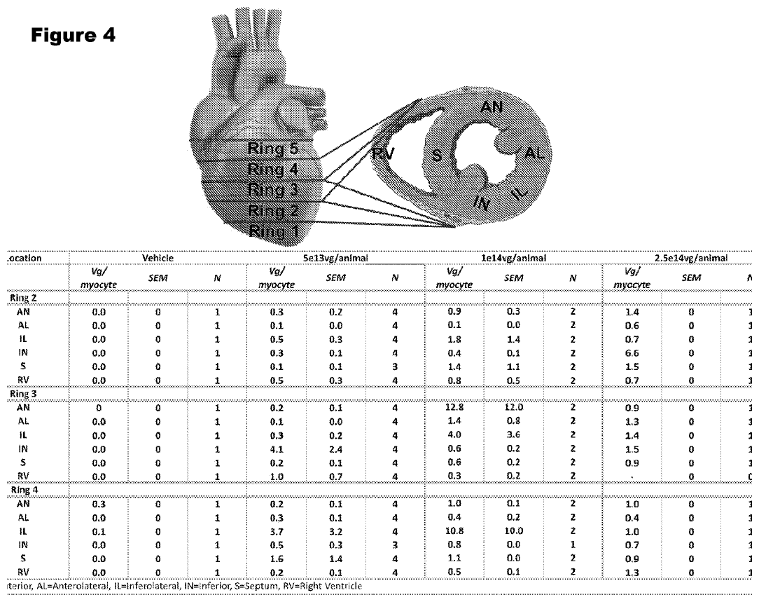

Eight weeks post dosing, 18 tissue sections from 3 short-axis circumferential segments of left ventricle tissue at 6 points along the long axis and right ventricular free wall were obtained as depicted in FIG. 4. The presence of AAV vector genomes (vg) was assessed by quantitative polymerase chain reaction and a standard curve of known copy numbers of transgene plasmid. Vector messenger RNA was assessed using quantitative polymerase chain reaction targeting vector complementary DNA and expressed as relative quantities to the 18S housekeeping gene. BAG3 expression was assessed using immunohistochemistry. Results are expressed as the mean SEM by group of relative quantities per animal, vg/mg of DNA, or vg/cardiomyocyte where possible (e.g., n≥1).

Measurements for vg or transcript more than 3 standard deviations (SDs) from the mean for that animal were excluded. To express vg/cardiomyocyte, each porcine cardiomyocyte was assumed to have 8 nuclei.

Genomic DNA (in vg/μg) were present in varying levels in all tissue samples. (Group A: 7,043±3,579 vg/μg; group B: 24,832±3,307 vg/μg; group C: 15,744 vg/μg, n=1) The mean quantity of vg was ≥1 per porcine cardiomyocyte at doses given in groups B and C (group A: 0.70±0.2; group B: 2.0±0.8; group C: 1.3).

Transcription of the BAG3 transgene was also measured in groups A, B, and C. Relative quantities of vector transcript (±SEM) normalized to 18S ribosomal RNA were vehicle group: 0.99; group A: 4.1±1.0; group B: 9.0±4.5; and group C: 8.5. BAG3 protein levels measured by immunohistochemistry remained unchanged.

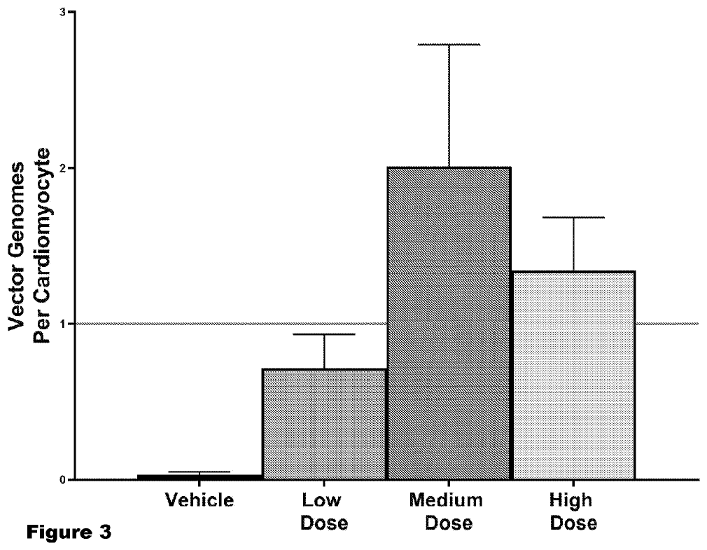

Results: The myocardium was safely and efficiently transduced with AAV9-BAG3 using RCSI in this study. Broad distribution of vector DNA/diploid genome was observed throughout the myocardium, including anterior left ventricle, anterior lateral left ventricle, inferior lateral left ventricle, inferior lateral ventricle, septum and right ventricle. On average, each cardiomyocyte had at least 1 copy of the introduced gene using total vg doses of 1×10¹⁴ vg (3.45×10¹² vg/kg) and 2.5×10¹⁴ vg (7.58×10¹² vg/kg). AAV vector transduction of the heart with the 1e14vg dose, after applying the exclusion criteria described above, appears to provide the greatest transduction of cells (FIG. 3). This study provides strong evidence that viral vectors, including AAV vectors, can deliver DNA to the heart. As expected, BAG3 protein levels were not altered presumably because BAG3 is autoregulated.

Given the presence and transcription of vector genome (vg) in the pig heart following administration of relatively low doses of AAV9-BAG3, RCSI can effectively provide expression of functional BAG3 protein, thereby exerting therapeutic effects in the setting of BAG3 deficiency such as haploinsufficiency. The variability in biodistribution seen across the myocardium supports the hypothesis that gene distribution may be influenced by inherent unevenness in myocardial strain.

In conclusion, safe delivery at relatively low doses of AAV9-BAG3 vg using catheter-based transvenous retrograde CS administration resulted in diffuse transduction of the myocardium. Using lower total vg doses using localized/targeted delivery via retrograde CS may translate to safety advantages in the clinic.

### Example 3

A subsequent dose toxicity study, which is ongoing, was performed using AAV9-BAG3 vector substantially as described in Example 2, except the AAV vector doses, volume administered, dose per minute were modified as described below. This study was undertaken using three AAV vector doses, 1e14vg, 2.5e14 and 5e14vg, per animal. The dose volume was reduced to 20 mL, which was administered over 20 minutes (1 mL/min). The results discussed below are from animals analyzed 3 months post infusion.

Methods: Healthy male Yucatan minipigs (weight: 30-40 kg) were sedated and randomized to receive one of 3 doses (1e14vg (n=6), 2.5e14vg (n=6), 2.5e14vg (n=6)) of rAAV2/9-BAG3 (vg=rAAV vector genomes) through the tip of a catheter placed under fluoroscopy in the coronary sinus (CS). Occlusion of the CS was confirmed by venography prior to injection. AAV9-BAG3 vector was infused in 20 mL of vehicle over 20 minutes with simultaneous balloon occlusion at the ostium. After administration of AAV9-BAG3 vector, occlusion was maintained for 5 minutes so the vector remained in the coronary sinus.

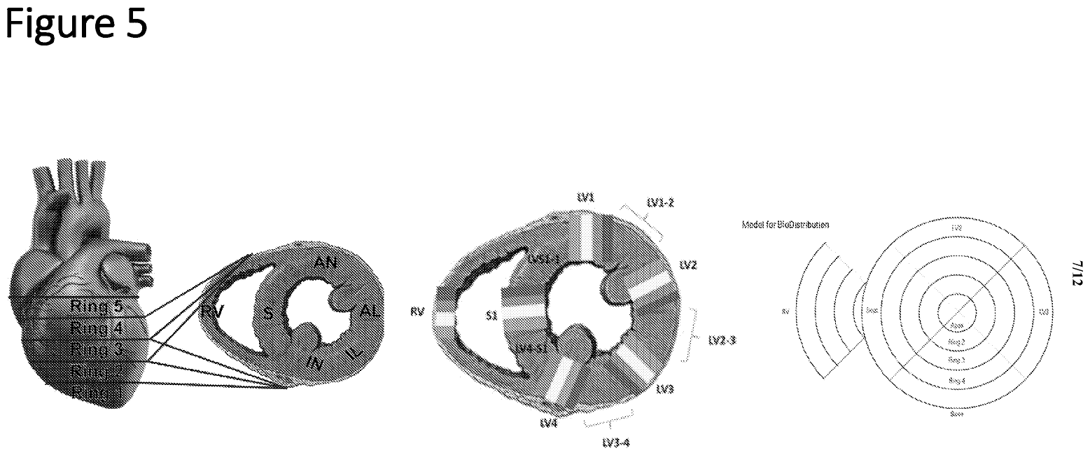

3 months post dosing, 18 tissue sections from 3 short-axis circumferential segments of left ventricle tissue at 6 points along the long axis and right ventricular free wall were obtained as depicted in FIG. 5. The presence of AAV vector genomes (vg) was assessed by quantitative polymerase chain reaction and a standard curve of known copy numbers of transgene plasmid. Vector messenger RNA was assessed using quantitative polymerase chain reaction targeting vector complementary DNA and expressed as relative quantities to the 18S housekeeping gene. BAG3 expression was assessed using immunohistochemistry.

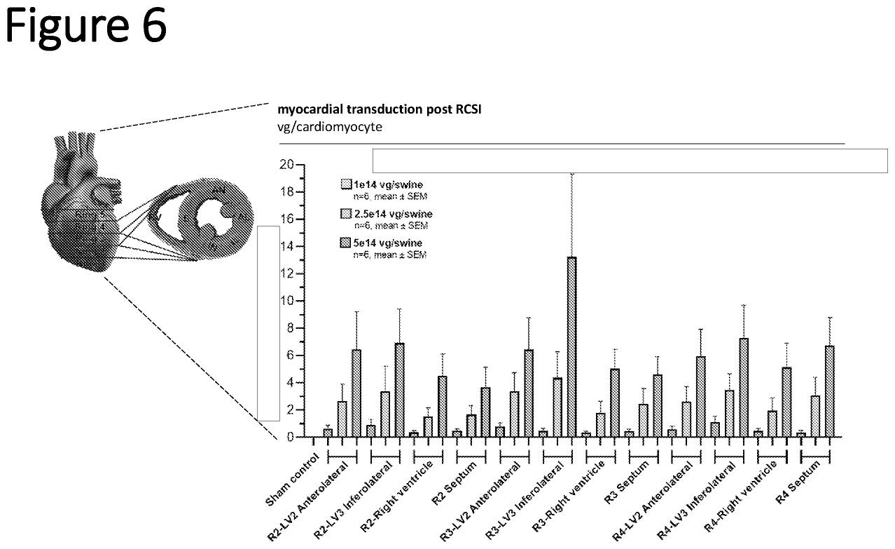

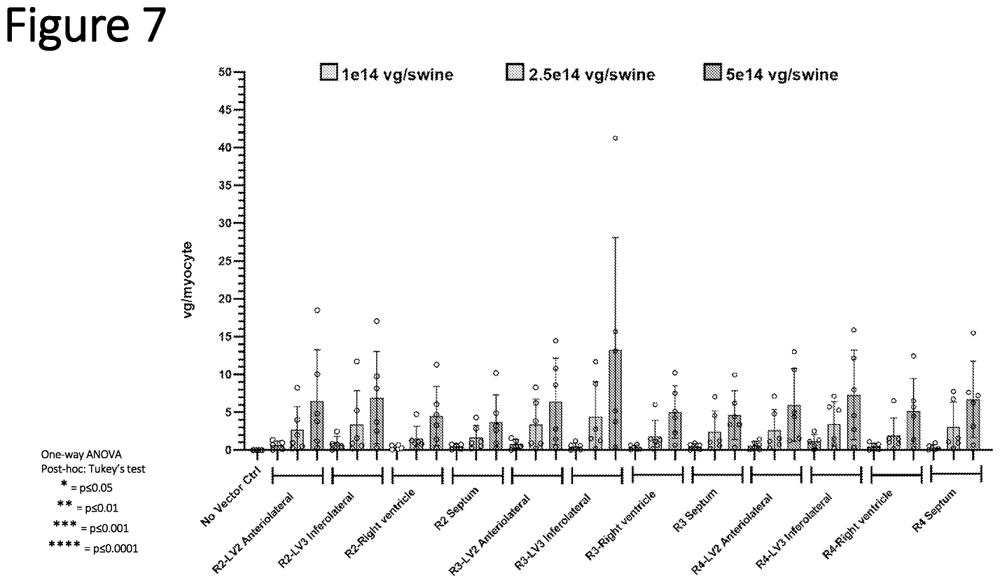

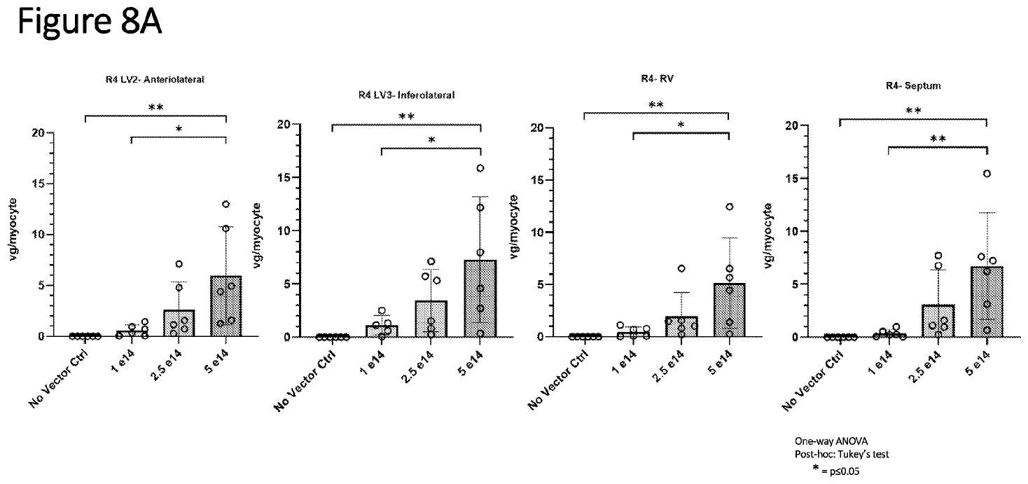

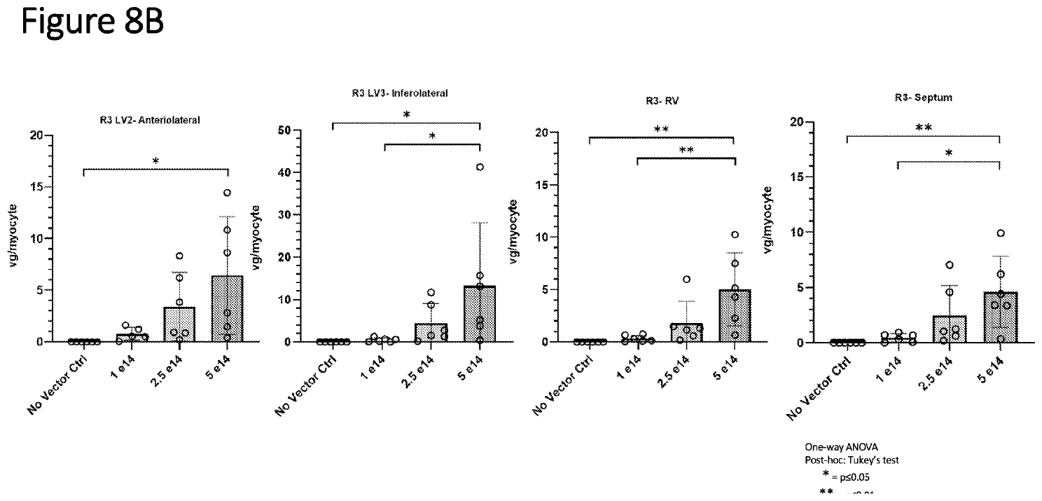

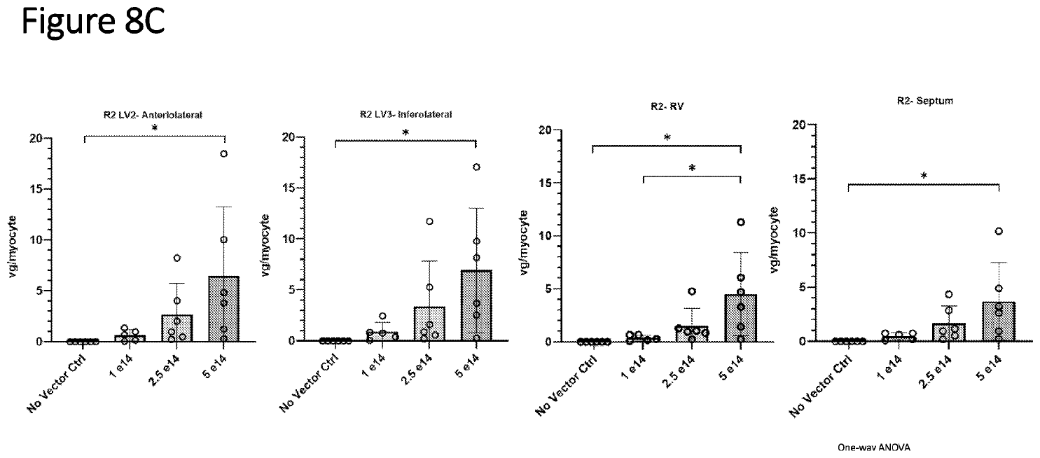

Results: The myocardium was safely and efficiently transduced with AAV9-BAG3 using RCSI in this subsequent dose toxicity study. Broad dose-dependent distribution of vector DNA/diploid genome was again observed throughout the myocardium, including anterior left ventricle, anterior lateral left ventricle, inferior lateral left ventricle, inferior lateral ventricle, septum and right ventricle (FIGS. 6-8).

BAG3 vector copies per nucleus (VCN) correlated with the dose administered. In particular, there were approximately 1, 3 and 6 vector copies per nucleus for the 1e14vg, 2.5e14 and 5e14vg doses, respectively (FIG. 6).

Each of these exemplary doses administered by way of retrograde coronary sinus infusion (RCSI) provides BAG3. AAV vector dose amounts can be extrapolated to humans based upon, for example, left ventricular mass and/or surface area.

additional durability (persistence) of BAG3 expression beyond 3 months will be assessed at 6 months (n=4) and 9 months (n=4) post infusion.

### Example 4

### Vector Copies Per Nucleus (VCN) Calculation Method:

### 1) Standard Curve and Transformation

a. Transgene plasmid digested with XhoI and PacI restriction enzymes, expected band purified and OD'd
b. The concentration in ng/μL (or μg/mL) of hBag3 plasmid DNA will be converted into copy numbers/μL according to following formula:

$$DNA in copies ( or molecules ) / μL = ( DNA in ng / μL \times {10}^{- 9} g / ng \times 6.022 \times {10}^{23} molecules / mole ) \text{} / ( 6048 bp \times 650 g / bp / mole )$$

i. This calculation is based on the length of hBag3 plasmid DNA at 6048 base pairs and the assumption that the average molecular weight of a base pair (bp) is 650 Daltons, Avogadro's number, 6.022×10²³ molecules/mole.
c. A standard curve (example curve shown below) with the following copies per reaction will be run: 10⁷, 10⁶, 10⁵, 10⁴, 10³, 10², 50, 25, 10, and 0. The standard curve equation will be used to determine copies per 1000 ng reaction (this number is N in equation below)

### 2) Conversion of Number of Copies/1 μg of DNA to Number of Copies/Diploid Genome

a. Size of the diploid genome of a Yucatan minipig is ˜5.33 pg DNA per nucleus.
b. 1000 ng/˜5.33 pg=˜187,600 nuclei (or cells, for those with a single nucleus)
c. In the standard curve there are 2 vector genomes for every copy of DNA standard when working with ssDNA, such as AAV
d. Equation for conversion of #copies (N)/1 ug DNA and convert to vg per nucleus:

$$i . [ 2 vg / copy ] \times [ \underline{N} copies / ug DNA ] \times [ \text{} 1 ug DNA / 187 , 600 nuclei ] = vg / nuclei$$

### 3) Examples

a. Example a from CRO—this is not using actual data

$$i . 2 , 000 , 000 copies / 1 ug * DNA / \sim 187 \text{} , 600 cells * 2 vg / copy = \sim 21.3 vg / cell$$

$$( diploid genome , single nuclei )$$

What is claimed is:

1. A method of delivering a therapeutic to the heart, comprising administering the therapeutic to a subject via retrograde coronary venous or sinus delivery, without occluding the left main coronary artery or without occluding antegrade flow, thereby delivering the therapeutic to the heart.

2. A method of delivering a therapeutic to the heart, comprising administering the therapeutic to a subject via retrograde coronary venous or sinus delivery, with occluding the left main coronary artery or with occluding antegrade flow, thereby delivering the therapeutic to the heart.

3. The method of claim 1 or 2, wherein the therapeutic comprises a nucleic acid or protein.

4. The method of claim 3, wherein the nucleic acid encodes a protein.

5. The method of claim 3 or 4, wherein the protein comprises BCL2-Associated Athanogene 3 (BAG3).

6. The method of claim 3, wherein the nucleic acid comprises an expression vector.

7. The method of claim 6, wherein the expression vector comprises a viral vector, eukaryotic or yeast vector.

8. The method of claim 7, wherein the viral vector comprises an adeno-associated virus (AAV) vector, adenovirus vector, lentiviral vector or retroviral vector.

9. The method of claim 8, wherein the AAV vector comprises a capsid or inverted terminal repeat from any one of the following AAV serotypes: AAV1, AAV2, AAV3, AAV4, AAV5, AAV6, AAV7, AAV8, AAV9, AAV10, AAV11 or AAV12.

10. The method of claim 6, wherein the expression vector comprises a promoter functional in heart.

11. The method of claim 10, wherein the promoter is cardiac specific.

12. The method of claim 6, wherein the expression vector comprises a promoter functional in heart and a BAG3 polynucleotide or cDNA sequence.

13. The method of any of claims 1-12, wherein the subject is human.

14. The method of any one of claims 1-13, wherein the subject or human is suffering from heart failure.

15. The method of any one of claims 1-14, wherein the subject or human is suffering from heart failure with reduced ejection fraction or heart failure with preserved ejection fraction.

16. The method of any one of claims 1-15, wherein the subject or human is suffering from familial dilated cardiomyopathy.

17. The method of any one of claims 1-15, wherein the subject or human is suffering from non-familial dilated cardiomyopathy.

18. The method of any one of claims 1-15, wherein the subject or human is suffering from ischemic heart disease or cardiomyopathy.

19. The method of any one of claims 1-15, wherein the subject or human is suffering from nonischemic heart disease or cardiomyopathy.

20. The method of any one of claims 1-15, wherein the subject or human is at risk of ischemia/reperfusion injury.

21. The method of any one of claims 1-20, wherein the subject or human is scheduled for or is a candidate for a vascular interventional or medical procedure that could be to ischemia/reperfusion injury.

22. The method of any one of claims 1-20, wherein the therapeutic is delivered to the heart by a catheter positioned proximal to the coronary sinus and distal to the great cardiac vein.

23. The method of any one of claims 1-20, wherein the therapeutic is delivered to the heart by a catheter positioned in or occluding the coronary sinus and distal to the origin of the great cardiac vein or the great cardiac vein.

24. The method of claim 23, wherein the vascular interventional or medical procedure comprises a procedure using a catheter, a stent, angioplasty, bypass surgery or coronary artery bypass graft.

25. The method of any one of claims 1-23, wherein the subject or human is scheduled for or is a candidate for peripheral vascular disease surgery.

26. The method of any one of claims 1-23, wherein the subject or human has a mutation in their endogenous BAG3 polynucleotide or polypeptide.

27. The method of any one of claims 1-23, wherein the subject or human has reduced expression or activity of endogenous BAG3 polynucleotide or polypeptide.

28. The method of any one of claims 1-27, wherein the therapeutic is administered to the subject or human via the great cardiac vein.

29. The method of any one of claims 1-27, wherein the therapeutic is administered via a catheter positioned proximal but distal to the great cardiac vein.

30. The method of any one of claims 1-27, wherein the therapeutic is delivered into the great cardiac vein, left azygous or any veins that feed off the coronary sinus.

31. The method of any one of claims 1-30, wherein the therapeutic is administered via infusion for a time up to about 20 minutes.

32. The method of any one of claims 1-30, wherein the therapeutic is administered at a rate of about 1 ml per minute to 5 ml per minute.

33. The method of any one of claims 1-30, wherein the therapeutic is administered at a rate of about 1 ml per minute.

34. The method of any one of claims 1-33, wherein the therapeutic is administered in a volume of about 10 ml to about 100 mL.

35. The method of any one of claims 1-33, wherein the therapeutic is administered in a volume of about 20 ml to about 75 mL.

36. The method of any one of claims 1-33, wherein the therapeutic is administered in a volume of about 30 ml to about 60 mL.

37. The method of any one of claims 1-33, wherein the therapeutic is administered in a volume of about 40 ml to about 50 mL.

38. The method of any one of claims 1-37, wherein the therapeutic is delivered to and/or expressed in one or more of anterior left ventricle, anterior lateral left ventricle, inferior lateral left ventricle, inferior lateral ventricle, septum or right ventricle.

39. The method of any one of claims 1-37, wherein the therapeutic is expressed throughout the heart.

40. The method of any one of claims 1-39, wherein the therapeutic reduces one or more symptoms of a cardiac disease.

41. The method of any one of claims 1-41, wherein the therapeutic reduces one or more symptoms of heart failure.

42. The method of any one of claims 1-41, wherein the therapeutic improves cardiac function or cardiac contractility.

43. The method of any one of claims 1-42, wherein the therapeutic increases left ventricle ejection fraction.

44. The method of any one of claims 1-43, wherein the therapeutic comprises a nucleic acid or protein.

45. The method of claim 43, wherein the nucleic acid comprises an expression vector.

46. The method of any one of claims 1-43, wherein the therapeutic comprises a viral vector.

47. The method of any one of claims 1-46, wherein the therapeutic or protein comprises BCL2-Associated Athanogene 3 (BAG3) or the nucleic acid or the expression vector encodes BAG3 or the viral vector comprises a nucleic acid or expression vector encoding BAG3.

48. The method of claim 46 or 47, wherein the viral vector is a eukaryotic or yeast vector.

49. The method of claim 46 or 47, wherein the viral vector comprises an adeno-associated virus (AAV) vector, adenovirus vector, lentiviral vector or retroviral vector.

50. The method of claim 49, wherein the AAV vector comprises a capsid or inverted terminal repeat from any one of the following AAV serotypes: AAV1, AAV2, AAV3, AAV4, AAV5, AAV6, AAV7, AAV8, AAV9, AAV10, AAV11 or AAV12.

51. The method of any one of claims 46-50, wherein the viral vector is administered at a dose from about 1×10¹¹ vg/kg to about 1.0×10¹⁴ vg/kg.

52. The method of any one of claims 46-50, wherein the viral vector is administered at a dose from about 1.0×10¹² vg/kg to about 0.5×10¹⁴ vg/kg.

53. The method of any one of claims 46-50, wherein the viral vector is administered at a dose from about 3.0×10¹² vg/kg to about 1.0×10¹³ vg/kg.

54. The method of any one of claims 46-50, wherein the viral vector is administered at a dose from about 3.0×10¹² vg/kg to about 9.0×10¹² vg/kg.

55. The method of any one of claims 46-50, wherein the viral vector is administered at a dose from about 3.0×10¹² vg/kg to about 8.0×10¹² vg/kg.

56. The method of any one of claims 46-50, wherein the viral vector is administered at a dose from about 3.0×10¹² vg/kg to about 5.0×10¹² vg/kg.

57. The method of any one of claims 2-56, wherein the occlusion is maintained for a period of about 30 seconds to about 20 minutes.

58. The method of any one of claims 2-56, wherein the occlusion is maintained for a period of about 1 minute to about 15 minutes.

59. The method of any one of claims 2-56, wherein the occlusion is maintained for a period of about 2 minutes to about 12 minutes.

60. The method of any one of claims 2-56, wherein the occlusion is maintained for a period of about 3 minutes to about 10 minutes.

61. The method of any one of claims 2-56, wherein the occlusion is maintained for a period of about 4 minutes to about 6 minutes.

62. The method of any one of claims 2-56, wherein the occlusion is maintained for a period of about 5 minutes.

63. The method of any one of claims 2-62, wherein the occlusion is at or near the ostium.
